(19) United States
(12) Patent Application Publication
(10) Pub. No.: US 2004/0121445 A1
(43) Pub. Date: Jun. 24, 2004

(54) Cell cultures

(76) Inventors: Fabien Marino, Toronto (CA); Aled Edwards, Toronto (CA); Akil Dharamsi, Richmond Hill (CA); Donald Awrey, Mississauga (CA)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/630,220

(22) Filed: Jul. 30, 2003

Related U.S. Application Data
(60) Provisional application No. 60/399,873, filed on Jul. 31, 2002.

(51) Int. Cl. C12N 1/21

(52) U.S. Cl. 435/252.3

(57) ABSTRACT

Disclosed herein are methods, media and other compositions relating to high density cell cultures. Also disclosed are exemplary methods for using high density cell cultures and exemplary products that may be obtained from high density cell cultures.

### RELATED APPLICATION INFORMATION

This application claims the benefit of priority to Provisional Patent Application No. 60/399,873, filed Jul. 31, 2002, which application is hereby incorporated by reference in its entirety.

### BACKGROUND

Improvements in nucleic acid cloning and sequencing have provided a wealth of gene sequence information. This gene sequence information has accelerated the pace of scientific inquiry relating to gene, protein and cellular functions. However, difficulties in characterizing protein characteristics and activities have created a bottleneck in the acquisition of knowledge about biological systems. Proteins are generally produced as linear amino acid chains that fold to adopt a three dimensional structure (or multiple interconverting conformations). The biological activities of a protein are typically determined by its structure and the position of amino acids within that structure; therefore structural information is an important aid in understanding protein function. Methods for deducing protein structure, such as nuclear magnetic resonance (NMR) and x-ray crystallography (XRC) demand relatively large quantities of purified protein. Other methods for analyzing proteins, including assays for probing the biochemical activities of proteins, also benefit from a ready source of purified protein.

In addition, many proteins are valuable therapeutic agents, industrial catalysts and research reagents. Erythropoietin and human growth hormone are examples of polypeptide therapeutics that have revolutionized the treatment protocols for a variety of diseases. Catalase and pectinase are examples of enzymes commonly used in industrial processes. Scientists employ purified proteins as standard reagents in a variety of cell biology experiments and cell culture systems. For example, recombinant growth factors, such as epidermal growth factor, hepatocyte growth factor, insulin and leukemia inhibitory factor, are widely used in the culture of mammalian cells and in the growing field of stem cell research.

Proteins are not easily produced in large quantities, and as a result, most purified proteins are expensive. Large scale protein production is typically performed using sophisticated culture systems termed “fermenters” and recombinant cell lines that are engineered to produce high levels of a desired protein. Fermenters permit close monitoring and manipulation of cell culture conditions to produce dense cell cultures and optimal protein production. However, fermenters are costly to acquire and maintain, and contribute significantly to the cost of protein production.

Alternate methods for production of proteins would benefit the biological research community, as well as patients, health care providers and many industry groups.

### BRIEF SUMMARY

The present invention provides novel methods and compositions for generating a high density cell culture. In various aspects, the invention provides methods for generating a high density cell culture in a simple culture flask. The invention also provides cell culture medium suitable for generating high density cell cultures. The invention further provides methods for producing polypeptides, including polypeptides suitable for structural and functional characterization by a variety of techniques, including, for example, affinity chromatography, mass spectrometry, NMR and x-ray crystallography. In certain embodiments, the invention provides methods for producing polypeptides comprising a label that facilitates structural characterization of the polypeptide by NMR or x-ray crystallography. In still further embodiments, the invention provides methods for high-throughput protein production.

In certain embodiments, the present invention makes it possible to generate high density cell cultures in simple culture flasks. This is in contrast to traditional high density cell culture methods involving fermented growths that may require specialized facilities, expensive equipment and reagents, and specially trained personnel. As a result, in certain embodiments, the invention provides a cost-effective and time-saving method that may optionally use novel compositions that allow for the high-throughput preparative scale production of polypeptides. In certain embodiments, the inventive methods will facilitate structural characterization and rational drug design of protein targets.

In one aspect, the invention provides a method of generating a high density cell culture, the method comprising:

(a) placing cells and a medium in a simple culture vessel, thereby generating a cell culture, the medium being suitable for growth of a high density cell culture; and

(b) operating the simple culture vessel to provide an oxygen transfer rate suitable for growth of a high density cell culture, thereby generating a high density cell culture, the high density cell culture having an optical density at a wavelength of 600 nm (OD₆₀₀) of 4 or greater.

In certain embodiments, the oxygen transfer rate is greater than 2.0 millimoles O₂/liter/minute.

In exemplary embodiments, cells used in accordance with the methods of the invention are bacterial cells. The bacterial cells may comprise an exogenously regulated expression construct, such as, for example, a construct comprising a lacI binding site operably linked to an expressible nucleic acid.

In certain embodiments, the cell culture may be contacted with an inducer, such as, for example IPTG. In exemplary embodiments, the cell culture may be contacted with an inducer when the culture has reached an OD₆₀₀ of 1, 2, 3, 4, or 5; or greater. In certain embodiments, the cell culture may be substantially maintained at a temperature higher than 25° C. prior to contacting the cell culture with the inducer and the cell culture is substantially maintained at a temperature lower than 25° C. after contacting the cell culture with the inducer.

In certain embodiments, the simple culture vessel is shaken on an orbital shaker table with an orbital diameter and shaking rate (revolutions per minute) suitable to provide an oxygen transfer rate greater than 2.0 millimoles O₂/liter/minute. In exemplary embodiments, the orbital shaker table has an orbital diameter of at least about one inch and the shaking rate is at least about 200 revolutions per minute. In one embodiment, the simple culture vessel is shaken at greater than 250 revolutions per minute. In one embodiment, the cell culture has a volume of less than 200 milliliters.

In certain embodiments, the optical density at a wavelength of 600 nm of a cell culture grown according to the methods described herein is 10 or greater, or 20 or greater. In certain embodiments, the cell culture has a volume of between 500 and 2000 milliliters. In other embodiments, the cell culture has a volume of less than 200 milliliters.

In certain embodiments, a medium used in accordance with the methods of the invention comprises:

(i) a carbon source selected from the group consisting of glycerol and glucose;

(ii) a complex organic material selected from the group consisting of tryptone, yeast extract, hydrolyzed casein and beef broth; and

(ii) a magnesium source.

In certain embodiments, the medium may further comprise:

iv) two or more metals selected from the group consisting of cobalt, manganese, copper, boron, molybdenum, zinc, iron, calcium, aluminum and nickel.

In an exemplary embodiment, a medium used in accordance with the methods of the invention comprises:

(i) tryptone at a concentration of 10-14 grams/L;

(ii) yeast extract at a concentration of 20-30 grams/L;

(iii) buffering salts at an initial pH of between 6 and 8;

(iv) magnesium sulfate at a concentration of between 0.5 and 2 mM;

(v) 0.5-5% glycerol; and

(vi) a metal mixture comprising cobalt, manganese, copper, boron, molybdenum, zinc, iron, calcium, aluminum and nickel.

In certain embodiment, the buffering salts may comprise potassium and phosphate, the phosphate at a concentration of between 100 and 200 mM.

In an exemplary embodiment, the metal mixture comprises: CoCl₂-6H₂O (4-8 μM), MnSO₄-5H₂O (20-40 μM), CuCl₂-2H₂O (4-8 μM), H₃BO₃ (6-10 μM), Na₂MoO₄-2H₂O (6-10 μM), ZnSO₄-7H₂O (4-8 μM), FeSO₄-7H₂O (75-125 μM), CaCl₂-2H₂O (40-100 μM), AlCl₃-6H₂O (2-6 μM), and NiCl₂-6H₂O (6-10 μM).

In various embodiments, the methods described herein may further comprising obtaining a partially purified or purified polypeptide composition from the high density cell culture. NMR and/or x-ray crystallography may optionally be used to determine structural information or determine a druggable region for a polypeptide produced in accordance with the methods and compositions described herein.

In various embodiments, a simple culture vessel used in accordance with the methods and compositions described herein may be a Tunac-type flask or a 96-well plate.

The invention also provides a high density cell culture, a partially purified polypeptide, or a purified polypeptide produced in accordance with the method described herein. In various embodiments, the polypeptides may be a therapeutically useful polypeptide, an industrially useful polypeptide or a polypeptide useful for research purposes.

In certain embodiments, a cell culture of the invention may be contacted with a label that may be incorporated into a polypeptide during growth of the high density cell culture. In one exemplary embodiment, the label is seleno-L-methionine. In other embodiments, the label is an isotopic label selected from the group consisting of potassium-40 (⁴⁰K), carbon-14 (¹⁴C), tritium (3H), sulphur-35 (³⁵S), phosphorus-32 (³²P), technetium-99m (^{99m}Tc), thallium-201 (²⁰¹Tl), gallium-67 (⁶⁷Ga), indium-111 (¹¹¹In), iodine-123 (¹²³I), iodine-131 (¹³¹I), yttrium-90 (⁹⁰Y), samarium-153 (¹⁵³Sm), rhenium-186 (₁₈₆Re), rhenium-188 (¹⁸⁸Re), dysprosium-165 (¹⁶⁵Dy), holmium-166 (¹⁶⁶Ho), hydrogen-1 (¹H), hydrogen-2 (²H), hydrogen-3 (³H), phosphorous-31 (³¹P), sodium-23 (²³Na), nitrogen-14 (¹⁴N), nitrogen-15 (¹⁵N), carbon-13 (¹³C) and fluorine-19 (¹⁹F). In still other embodiments, the label is a heavy atom label selected from the group consisting of cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium.

In an exemplary embodiment, a medium used in accordance with the methods of the invention comprises:

i) a carbon source selected from the group consisting of glycerol and glucose;

ii) a basal nutrient source; and

iii) a label selected from the group consisting of an isotopic label, a heavy atom label, and seleno-L-methionine.

In various embodiments, the medium may further comprise one or more amino acids.

In an exemplary embodiment, a medium used in accordance with the methods of the invention comprises: 0.04-4% glucose, NH₄Cl at a concentration of 20-60 mM, KH₂PO₄ at a concentration of 20-60 mM, Na₂HPO₄ at a concentration of 75-115 mM, Na₂HPO₄-7H₂O at a concentration of 75-115 mM, MgSO₄ at a concentration of 0.5-4 mM, FeSO₄ at a concentration of 70-110 mM, CaCl₂ at a concentration of 80-120 μM, one or more amino acids but not methionine, vitamins, and seleno-L-methionine at a concentration of 100-300 μM.

In another aspect, the invention provides a method of culturing cells, the method comprising:

(a) placing cells and a medium in a simple culture vessel, thereby generating a cell culture, the medium comprising:

(i) tryptone at a concentration of 10-14 grams/L;

(ii) yeast extract at a concentration of 20-30 grams/L;

(iii) buffering salts at a pH of between 6 and 8; and

(iv) MgSO₄ at a concentration of between 0.5 and 2 mM; and

(b) operating the simple culture vessel to provide an oxygen transfer rate greater than 2.0 millimoles O₂/liter/min.

In certain embodiments, the medium further comprises:

v) 0.5-5% glycerol;

vi) a metal mixture comprising: cobalt, manganese, copper, boron, molybdenum, zinc, iron, calcium, aluminum and nickel; and

vii) vitamins.

In another aspect, the invention provides a method for producing a labeled polypeptide, the method comprising:

a) placing cells and a medium in a simple culture vessel, thereby generating a cell culture, the medium comprising:

i) a carbon source selected from the group consisting of glycerol and glucose;

ii) a basal nutrient source; and

iii) a label selected from the group consisting of an isotopic label, a heavy atom label, and seleno-L-methionine; and

b) operating the simple culture vessel to provide an oxygen transfer rate greater than 2.0 millimoles O₂/liter/min.

In an exemplary embodiment, a medium that may be used in accordance with the methods of the invention comprises: 0.04-4% glucose, NH₄Cl at a concentration of 20-60 mM, KH₂PO₄ at a concentration of 20-60 mM, Na₂HPO₄ at a concentration of 75-115 mM, Na₂HPO₄-7H₂O at a concentration of 75-115 mM, MgSO₄ at a concentration of 0.5-4 mM, FeSO₄ at a concentration of 70-110 mM, CaCl₂ at a concentration of 80-120 μM, one or more amino acids but not methionine, vitamins, and seleno-L-methionine at a concentration of 100-300 μM.

In another aspect, the invention provides a method for high-throughput production of polypeptides, the method comprising:

(a) obtaining a plurality of cell lines, each cell line comprising an exogenously controlled expression construct for expressing a nucleic acid encoding a polypeptide;

(b) generating a plurality of cell cultures by placing in separate simple culture vessels medium suitable for growth of a high density cell culture, and cells of one of the plurality of cell lines;

(c) operating the simple culture vessels to provide an oxygen transfer rate suitable for growth of a high density cell culture, thereby obtaining a high density cell culture having an OD₆₀₀ of 4 or greater; and

(d) obtaining a purified polypeptide composition from a plurality of the high density cell cultures, thereby obtaining purified polypeptide compositions.

In certain embodiments, each cell line comprises an exogenously controlled expression construct for expressing a nucleic acid encoding a polypeptide, wherein each polypeptide is a polypeptide of the proteome of a subject organism. In other embodiments, each cell culture is contacted with an inducer at an OD₆₀₀ of 1 or greater. Each cell culture may be contacted with an inducer at approximately the same time after generating the cell culture.

In another aspect, the invention provides a cell culture medium comprising:

(a) tryptone at a concentration of 10-14 grams/L;

(b) yeast extract at a concentration of 20-30 grams/L;

(c) buffering salts at an initial pH between 6 and 8;

(d) a metal mixture comprising: cobalt, manganese, copper, boron, molybdenum, zinc, iron, calcium, aluminum and nickel; and

(e) a magnesium source.

In an exemplary embodiment, the magnesium source is MgSO₄.

In various embodiments, the medium further comprises 0.5-5% glycerol.

In other embodiments, the metal mixture comprises: CoCl₂-6H₂O (4-8 EM), MnSO₄-5H₂O (20-40 μM), CuCl₂-2H₂O (4-8 μM), H₃BO₃ (6-10 μM), Na₂MoO₄-2H₂O (6-10 μM), ZnSO₄-7H₂O (4-8 μM), FeSO₄-7H₂O (75-125 μM), CaCl₂-2H₂O (40-100 μM), AlCl₃-6H₂O (2-6 μM), and NiCl₂-6H₂O (6-10 μM).

In other embodiments, the buffering salts comprise potassium and phosphate, the phosphate at a concentration of between 100 and 200 mM.

In certain embodiments, the invention provides a dry composition that, when mixed with an appropriate volume of water, provides a cell culture medium as described further herein. The invention also provides for a concentrated medium that, when mixed with an appropriate volume of water, provides a cell culture medium as described further herein.

In another aspect, the invention provides a metal mixture comprising water and at least eight elements selected from the group consisting of: cobalt, manganese, copper, boron, molybdenum, zinc, iron, calcium, aluminum and nickel. In certain embodiments, the metal mixture has a pH between 6 and 8. In an exemplary embodiment, the metal mixture comprises: CoCl₂-6H₂O (4-8 μM), MnSO₄-5H₂O (20-40 μM), CuCl₂-2H₂O (4-8 μM), H₃BO₃ (6-10 μM), Na₂MoO₄-2H₂O (6-10 μM), ZnSO₄-7H₂O (4-8 μM), FeSO₄-7H₂O (75-125 μM), CaCl₂-2H₂O (40-100 EM), AlCl₃-6H₂O (2-6 μM), and NiCl₂-6H₂O (6-10 μM).

In certain embodiments, the invention provides a dry composition that, when mixed with an appropriate volume of water, provides a metal mixture as described herein. In other embodiments, a concentrated metal mixture is provided that, when mixed with an appropriate volume of water, provides a metal mixture as described further herein.

The embodiments and practices of the present invention, other embodiments, and their features and characteristics, will be apparent from the description, figures and claims that follow, with all of the claims hereby being incorporated by this reference into this Summary.

### DETAILED DESCRIPTION

To provide an overall understanding, certain illustrative embodiments will now be described; however, it will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore unless otherwise specified, features, components, modules, and/or aspects of the disclosed inventions can be combined, separated, interchanged, and/or rearranged without departing from the disclosed systems, compositions, or methods.

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “basal nutrient source” refers to a nutrient source provided for by a minimal medium. In an exemplary embodiment, a basal nutrient source refers to a nutrient source suitable for growth of a bacterial strain, such as E. coli, including a carbon source (e.g., glucose or glycerol), a nitrogen source (e.g., NH₄Cl), a sulfur source (e.g., MgSO₄), a phosphorus source (e.g., Na₂HPO₄ and KH₂PO₄), a source of sodium, potassium, magnesium, and chloride, and a calcium source (e.g., CaCl₂).

The terms “cell culture” or “culture” include any combination of cells and medium. The cells need not be actively growing.

The terms “comprise” and “comprising” are used in the inclusive, open sense, meaning that additional elements may be included.

The term “culture vessel” includes any vessel suitable for holding a liquid cell culture. Many culture vessels are known in the art. Exemplary culture vessels include fermenters, Erlenmeyer flasks, baffled flasks, Tunac-type flasks (e.g. the Tunac flask), 96-well plates (or other multi-chambered systems), beakers, bags, test tubes, spinner flasks, Fernbach flasks, etc. A “simple culture vessel” is a culture vessel that is not equipped for providing a partial pressure of oxygen that substantially exceeds that of the room (or other general surroundings) in which the simple culture vessel is located. Examples of simple culture vessels include Erlenmeyer flasks, baffled flasks, Tunac-type flasks, 96-well plates, beakers, bags, test tubes, spinner flasks, Fernbach flasks, etc. Many simple culture vessels do not have any means for mixing the culture. Such vessels may be termed “externally agitated culture vessels”, and cultures contained in externally agitated culture vessels are generally mixed by attaching the flask to a device that agitates the culture, for example by providing orbital motion, back and forth motion or rocking motion. In certain embodiments, external agitation is provided by attaching the flask to an orbital shaker table.

A “fusion protein” or “fusion polypeptide” refers to a chimeric protein as that term is known in the art and may be constructed using methods known in the art. In various embodiments, fusion proteins comprise two or more polypeptide sequences that optionally may be linked in frame. A “fusion gene” refers to a nucleotide sequence encoding a fusion protein.

The term “gene” refers to a nucleic acid comprising an open reading frame encoding a polypeptide having exon sequences and optionally intron sequences. The term “intron” refers to a DNA sequence present in a given gene which is not translated into protein and is generally found between exons.

The term “including” is used herein to mean “including but not limited to”. “Including” and “including but not limited to” are used interchangeably.

An “inducer” includes any manipulation of the conditions of a cell culture that substantially and predictably affects the expression from an exogenously regulated expression construct. An exogenously regulated expression construct is generally designed to be regulated by one or more specific inducers. Exemplary inducers include, for example, isothiopyranogalactoside (IPTG), temperature (e.g., a shift in temperature so as to increase or decrease the temperature), xylose, tetracycline, etc.

The term “medium,” as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. The term “medium” also refers to material that is intended for use in a cell culture, even if it has not yet been contacted with cells. Similarly, a powder mixture that, when mixed with water or other liquid, becomes suitable for cell culture, may be termed a “powdered medium”. The term “minimal medium” includes media that support growth of the wild-type form of a species but do not support growth of one or more auxotrophic strains of that species. A supplemented minimal medium is a minimal medium that includes one or more additional substances in order to support growth of an auxotrophic strain. “Defined medium” or “defined minimal medium” refer to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD₆₀₀ of 3 or greater when other conditions (such as temperature and oxygen transfer rate) permit such growth.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

“Operating” as used in reference to a simple culture vessel includes any appropriate manipulation that is useful for creating desirable conditions for a cell culture. For example, the oxygen transfer rate may be increased by causing mixing of the culture. In a vessel such as an Erlenmeyer or Tunac-type flask, mixing is generally provided by physically moving the flask to cause agitation of the cell culture inside. In a vessel containing internal impellers, mixing may be provided by causing the impellers to move.

“Optical density at a wavelength of 600 nm” or “OD₆₀₀” refers to a standard measure of cell density in a culture obtained by calculating the absorbance of a 1 cm pathlength of culture at a 600 nm wavelength of light, minus the absorbance of a 1 cm pathlength of the medium (without cells).

The term “or” as used herein should be understood to mean “and/or”, unless the context clearly indicates otherwise.

The phrase “oxygen transfer rate” is art recognized. Oxygen transfer rate values described herein may be calculated using, for example, the sulfite oxidation procedure described in U.S. Pat. No. 4,665,035.

The term “polypeptide”, and the terms “protein” and “peptide” which are used interchangeably herein, refers to a polymer of amino acids. Exemplary polypeptides include gene products, naturally-occurring proteins, homologs, orthologs, paralogs, fragments, and other equivalents, variants and analogs of the foregoing. Polypeptides may also include one or more modifications such as, for example, a lipid moiety, a phosphate, a sugar moiety, etc.

The term “proteome” refers to the proteins expressed by a genome. Proteome also encompasses a “functional proteome” that represents the proteins present in a sample under a given set of conditions. In various embodiments, a proteome sample may represent the proteins present in a cell, tissue, organ, biological sample (such as, blood, serum, urine, saliva, biopsy specimen, etc.), organism, or species of interest. The polypeptides present in a sample may vary based on a range of factors that may have occurred prior to obtaining the protein sample, such as life cycle, disease state, and/or environmental conditions like temperature, oxygen tension, osmolarity, pH, nutrient availability, presence of a test compound, etc. The “proteome of a subject organism” is the set of proteins produced or predicted to be produced by an organism of interest.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis and mass-spectrometry analysis.

The term “recombinant nucleic acid construct” includes any combination of nucleic acid sequences that was generated by a technique of molecular biology. For example, recombinant nucleic acids may include nucleic acid sequences fused together by ligation, by polymerase chain reaction, by integration of a nucleic acid into a chromosome or episome of a cell (e.g. by transposon, by homologous recombination, by non-homologous recombination, by phage insertion, etc.). A “recombinant nucleic acid expression construct” includes any recombinant nucleic acid construct that comprises an expressible nucleic acid and appropriate cis-acting sequences to permit expression of the nucleic acid (e.g. an initiation signal, promoter, enhancer, regulator, etc.). An exemplary recombinant nucleic acid expression construct is a plasmid carrying a gene operably linked to an IPTG-inducible promoter. Another exemplary recombinant nucleic acid expression construct is an enhancer inserted into the genome next to the endogenous gene encoding the desired protein. An “exogenously regulated expression construct” is a recombinant nucleic acid expression construct wherein the rate of generation, degradation or accumulation of expressed nucleic acid is at least partially controlled by an external factor (i.e. “inducer”) that may be readily provided by one of skill in the art. An exogenously regulated expression construct is generally designed to be at least partially controlled by one or more specific inducers. For example, a P_{lac} promoter may be regulated by exogenously supplied galactose, or variants of galactose, such as isothiopyranogalactoside (IPTG). Promoters regulated by temperature sensitive transcription factors may be induced by changes in temperature. P_{xyl} promoters may be induced with xylose, and many other exogenously regulated expression constructs, along with the appropriate inducers, are known in the art.

The term “recombinant protein” refers to a protein which is produced by recombinant DNA techniques. For example, a nucleic acid encoding a polypeptide may be inserted into a suitable expression vector which is in turn used to transform a host cell suitable for expression of the polypeptide. In various embodiments, the term recombinant protein includes proteins having an amino acid sequence of a native protein or polypeptides similar thereto generated by mutations including substitutions and deletions of the naturally occurring protein. In other embodiments, recombinant proteins include polypeptide fusions comprising an amino acid sequence of a native protein, or fragments or derivatives thereof, fused to a heterologous polypeptide. Exemplary fusions proteins comprise a sequence that increases the solubility and/or facilitates purification, identification, detection, and/or structural characterization of another polypeptide to which it is fused.

The term “Tunac-type flask” refers generally to any simple culture vessel that is substantially radially symmetrical about a central axis and has a substantially symmetrically spaced array of annular baffles and peripheral baffles located at the inner surface of the base of the vessel. Exemplary Tunac-type flasks are described in U.S. Pat. No. 4,665,035. Exemplary Tunac-type flasks include the Tunair flasks, commercially available from Shelton Scientific (Shelton, Conn.).

2. Culture Methods

In certain aspects, the invention relates to a method for generating a high density cell culture. In certain embodiments, a high density cell culture is a cell culture that has an optical density at a wavelength of 600 nm (OD₆₀₀) of at least 4 or an OD₆₀₀ of at least 5, at least 10, at least 15, at least 20, at least 30 or, optionally, at least 40. OD₆₀₀ is a standard measure of cell density in a culture obtained by calculating the absorbance of a 1 cm pathlength of culture at a 600 nm wavelength of light, minus the absorbance of a 1 cm pathlength of the medium (without cells). Different pathlengths may be employed, as well as different wavelengths. The OD₆₀₀ may be inaccurate in cultures having a density higher than about 0.7, but a more accurate measurement may be obtained by diluting the culture prior to the spectrophotometric measurement. Other spectrophotometric and non-spectrophotometric methods for assessing culture density, such as transmission measurements and dilution plating, are known in the art, and it is generally possible to calibrate different methods against each other so that the measurements may be compared.

In certain embodiments, methods for generating a high density cell culture disclosed herein may employ a simple culture vessel. A simple culture vessel is a culture vessel that is not equipped for providing a partial pressure of oxygen that exceeds that of the room (or other general surroundings) in which the simple culture vessel is located. Examples of simple culture vessels include Erlenmeyer flasks, baffled flasks, Tunac-type flasks, 96-well plates, beakers, bags, test tubes, spinner flasks, Fernbach flasks, etc. Exemplary Tunac-type flasks are described in U.S. Pat. No. 5,075,234 and may be purchased from Shelton Scientific (Shelton, Conn.) under the name “Tunair” flask.

In certain embodiments, it may be desirable to manipulate the rate at which one or more gases dissolve into a culture (the gas transfer rate). Many cells grow more rapidly or to a greater density if the culture is supplied with an increased oxygen transfer rate. Alternatively, certain cells, particularly anaerobic cells, grow more rapidly or to a greater density if the culture is depleted of oxygen. The transfer rate of a gas into a liquid culture is determined by a host of variables, including the surface area between the gas and the liquid, the relative movement of liquid and gas past each other, the temperature, the partial pressure of the gas already dissolved in the liquid, the partial pressure of the gas in the gaseous phase, etc. These variables may be manipulated in a variety of ways. For example, fermenters are typically equipped with a pressurized supply of oxygen, and the culture is maintained at a high pressure so that a very high oxygen transfer rate and partial pressure of dissolved oxygen is obtained. In methods that employ a simple culture vessel, oxygen transfer rate may be manipulated by, for example, selecting a vessel shape and culture volume that provide a desired surface area, by agitating the culture, or by placing the simple culture vessel in a room or other enclosed space that is supplied with a partial pressure of oxygen that is greater or less than the average atmospheric partial pressure of oxygen. Agitation of the culture may be achieved by using a simple culture vessel that is equipped with an agitation element, such as a set of impellers. Many simple culture vessels do not have any integrated means for agitating the culture, and these are termed “externally agitated culture vessels”. A variety of systems for agitating an externally agitated culture vessel are available. For example, a vessel may be attached to a device that agitates the culture, such as a device that provides one or more of orbital motion (horizontal or vertical), back and forth motion or rocking motion. In certain embodiments, external agitation is provided by attaching the vessel to an orbital shaker table. Optionally, a method of the invention may employ an orbital shaker table with an orbital radius of one inch and a rotational speed of 200 to 300 revolutions per minute.

Generally, agitation speed and orbit affect both aeration and the mixing of the culture. Greater aeration increases oxygen transfer rate. The size of the orbit affects gas transfer rates linearly and the speed has a square-law relationship. Therefore, if the size of the orbit doubles, say from ½″ to 1,″ the gas transfer rate should double. If the speed doubles, the gas transfer should quadruple. Of course, these are general rules and certain flask shapes or other conditions may cause deviation from predicted effects.

Oxygen transfer rate (also sometimes termed “aeration rate”) may be measured by any of a variety of methods known in the art. One general approach to assessing the oxygen transfer rate involves operating the cell culture system under conditions similar to those used for actual cultures, but the culture vessel contains a liquid having a reagent that readily reacts with oxygen to create a measurable effect. By tracking the amount of reagent that reacts with oxygen over time in the culture system, an oxygen transfer rate can be determined. The oxygen transfer rate reflects the rate at which oxygen may be supplied to cells in culture. Oxygen absorption rates are commonly expressed in terms of millimoles of oxygen/liter/minute. For example, oxygen transfer rates may be measured by the sulfite oxidation method described in U.S. Pat. No. 4,665,035.

In certain embodiments, a high density cell culture is generated by placing cells and liquid media in a simple culture vessel and providing an oxygen transfer rate of at least 2.0 millimoles O₂/liter/minute, or at least 3.0 millimoles O₂/liter/minute, or at least 4.0 millimoles O₂/liter/minute, or at least 5.0 millimoles O₂/liter/minute, or at least 7.0 millimoles O₂/liter/minute. In certain embodiments, the oxygen transfer rate is at least equivalent to that obtained by agitating 100 mL of aqueous culture in a 300 mL Tunair flask (Shelton Scientific) on a horizontal orbital shaker having an orbital diameter of at least 1 inch and at a speed of at least 200 revolutions per minute, or optionally at least 250 or 300 rpm. Exemplary orbital shakers include, for example, Innova 4330 from New Brunswick Scientific Co., Inc., Edison, N.J. or GyroMax 767R from Amerex Instruments, Inc., Lafayette, Calif. In certain embodiments, a high density cell culture is obtained by placing cells and medium having a total volume of 150 mL or less (optionally, 100 mL or less) in a Tunac-type vessel, wherein the vessel can hold a total volume of about 250 mL or greater (optionally, 300 mL or greater), and providing an oxygen transfer rate of at least about 2.0 millimoles O₂/liter/minute, or at least 3.0 millimoles O₂/liter/minute, or at least 4.0 millimoles O₂/liter/minute, or at least 5.0 millimoles O₂/liter/minute, or at least 7.0 millimoles O₂/liter/minute. Optionally, agitation is provided by an orbital shaker having an orbital diameter of at least 1 inch and operated at a speed of at least 200 rpm, at least 250 rpm or at least 300 rpm. Optionally, the cells and medium have a total volume of 5 liters or less (optionally, 2 liters or less, 1 liter or less, or 500 mL or less) and the Tunac-type vessel has a total holding volume of at least three times the volume of cells and medium. In certain embodiments, cells are cultured in a culture system that is operated so as to provide an oxygenation capacity greater than 5 millimoles O₂/liter/minute, optionally greater than 10 or greater than 15 millimoles O₂/liter/minute.

The temperature for the cell culture may be selected as appropriate for the cell type and the intended use. In general, higher temperatures promote more rapid growth, but excessively high temperatures may be damaging. For example, a variety of laboratory bacterial strains, such as Escherichia coli and Bacillus subtilis have optimal growth rates in the range of 30° C. to 45° C., and particularly about body temperature (i.e. 37° C.). Where the cell culture is to be used for production of a polypeptide, it may be desirable to use a cooler temperature, such as a temperature below room temperature (i.e. 25° C.), or below 20° C., and optionally a temperature of about 15° C. may be used. It may also be desirable to use different temperatures at different times during the culture (see below, regarding protein production).

3. Media

Various media may be used for cell culture methods disclosed herein. In describing various media herein, it should be understood that media reagents measured in terms of volume should be measured at room temperature and atmospheric pressure unless otherwise specified. For convenience, and in accord with the conventions of the art, media are described in terms of the components that are mixed together to make the medium. Interactions between components, as well as dissociations and reassociations of components, are not taken into account. For example, a medium may be said to contain 50 mM potassium chloride, even if, after the salt is mixed into the medium, the medium actually contains 50 mM potassium ion and 50 mM chloride ion and essentially none of the original undissociated salt.

In certain embodiments, a medium for use in generating a high density cell culture will be a rich medium comprising one or more complex biological extracts, such as, for example, yeast extract, beef extract, casamino acids, peptone and tryptone (all available from Voigt Global Distribution LLC, Kansas City, Mo.). In other embodiments, a medium for use in generating a high density cell culture may be a minimal medium such as Bio-express 1000 (CIL), Bio-express min (Cambridge Isotope Laboratories, Inc., Andover, Mass.), Minimal Medium M9, Silantes (VLI Research, Inc., Malvern, Pa.), Martek 9 (Martek Biosciences Corp., Columbia, Md.), etc. In still other embodiments, a supplemented minimal media that has been supplemented with one or more additional substances to support growth of a cell culture may be used in accordance with the methods and compositions described herein.

In certain embodiments, a medium to be employed in a method disclosed herein may comprise one or more buffering salts in addition to whatever buffering capacity may be provided by any complex biological extracts. In general, buffering salts are an acid or base having a pKa near (optionally within 1-2 pH units) of the desired pH of the medium. Exemplary buffing salts include phosphate salts (e.g. sodium phosphate, potassium phosphate), Tris (usually as a halide salt, such as Tris-Cl), the Good buffers (e.g. HEPES, MES, etc.) and many amino acids that may be used as buffering salts. Buffering salts may be added to media as an acid or base and then, if necessary, adjusted to the desired pH by using a strong acid or base (e.g. hydrochloric acid, phosphoric acid, sulfuric acid, sodium hydroxide, potassium hydroxide, etc.). Alternatively, buffer salts may be added in the salt form, such as NaH₂PO₄ (monosodium phosphate), KH₂PO₄, K₂HPO₄, etc. It may be desirable to mix multiple salt and acid or base forms of a particular buffer salt system. The pH that the medium is adjusted to will determine the concentration of the various ionic and non-ionic forms of the buffer salts. For example, a potassium phosphate buffered aqueous medium will contain PO₄³⁻, HPO₄²⁻, H₂PO⁻, H₃PO₄ and K⁺ in varying concentrations depending on the pH. An exemplary buffer salt system comprises between about 50 and 300 mM potassium or sodium phosphate, optionally between about 100 and 200 mM potassium or sodium phosphate, and, as a further option, approximately 150 mM potassium or sodium phosphate.

In certain embodiments, a medium to be employed in a method disclosed herein comprises one or more carbon sources in addition to whatever carbon sources may be provided by any complex biological extracts. Exemplary types of carbon sources include simple sugars, complex sugars, alcohols, lipids and organic acids. Exemplary carbon sources include glucose and glycerol. Exemplary media may contain about 0.5 to 5% glycerol (percentage calculated as volume of glycerol per total volume of medium, usually by making a 50% glycerol stock solution of equal volumes water and glycerol and then calculating further dilutions based on the volume of 50% glycerol used per total volume calculations). In an exemplary embodiment, glycerol is used as a carbon source for cell cultures expressing a desired gene from a lacI-regulated promoter.

In certain embodiments, a medium to be employed in a method disclosed herein comprises one or more magnesium sources in addition to whatever magnesium may be provided by any complex biological extracts. A magnesium source may be any magnesium-containing compound that is soluble in the medium and has little or no undesirable effect on the cell culture. Exemplary magnesium sources include magnesium sulfate (MgSO₄), magnesium chloride (MgCl₂), magnesium hydroxide (Mg(OH)₂, although this basic magnesium source may require balancing with an acid), etc. Exemplary media comprise sufficient amounts of one or more magnesium sources to provide about 0.1-10 mM magnesium ions (Mg²⁺), and optionally about 1-2 mM Mg²⁺. For example, a medium may comprise about 0.1-10 mM MgSO₄, and optionally about 1-2 mM MgSO₄.

In certain embodiments, a medium to be employed in a method disclosed herein comprises one or more metal sources in addition to whatever metals may be provided by any complex biological extracts, magnesium sources or buffering salts. The one or more metal sources may be referred to as a metal mix, whether the one or more metal sources are added directly to the medium or mixed with water to form a metal solution prior to addition to the medium. It may be desirable to include one or more of the following metals in a medium in such valency states as may be appropriate: cobalt (Co), manganese (Mn), copper (Cu), boron (B), molybdenum (Mo), zinc (Zn), iron (Fe), calcium (Ca), aluminum (Al) and nickel (Ni). Optionally, a medium comprises at least two, at least three, at least four, at least five, at least six, at least seven, at least eight, at least nine, or all ten of the foregoing metals in some form. Most metals occur in a variety of valence states. In an exemplary embodiment, metals may be used in the following valence states: cobalt(II), manganese(II), copper(II), molybdenum(VI), zinc(II), iron(II), calcium(II), aluminum(III) and nickel(II). Boron may be supplied as the borate oxyanion, (BO₃)³⁻. Mo (VI) may be supplied as the molybdate oxyanion, (MoO₄)²⁻. Optionally, a medium comprises at least two, at least three, at least four, at least five, at least six, at least seven, at least eight, at least nine, or all ten of the foregoing metals in the preferred valence state. When not supplied as oxyanions (or other anionic molecular forms), the metals may be supplied as salts with one or more negatively charged counter ions, such as hydroxyl (OH)⁻, sulfate (SO₄)²⁻, and chloride Cl⁻. Metals supplied as oxyanions are generally supplied as salts with one or more positively charged counter ion, such as hydrogen H⁺, lithium Li⁺, sodium Na⁺, potassium K⁺, and ammonium (NH₄)⁺. Exemplary metal sources include CoCl₂, MnSO₄, CuCl₂, H₃BO₄, Na₂MoO₄, ZnSO₄, FeSO₄, CaCl₂, AlCl₃ and NiCl₂. Optionally, a medium comprises at least two, at least three, at least four, at least five, at least six, at least seven, at least eight, at least nine, or all ten of the foregoing metals as the aforementioned salt. The metal source molecule may also be a hydrate with one or more water molecules, such as, for example: CoCl₂-6H₂O, MnSO₄-5H₂O, CuCl₂-2H₂O, Na₂MoO₄-2H₂O, ZnSO₄-7H₂O, FeSO₄-7H₂O, CaCl₂-2H₂O, AlCl₃-6H₂O and NiCl₂-6H₂O. Optionally, a medium comprises at least two, at least three, at least four, at least five, at least six, at least seven, at least eight, at least nine, or all ten of the foregoing metals in the aforementioned hydrate. In certain embodiments a medium contains an exemplary metal mix comprising CoCl₂-6H₂O 4-8 μM, MnSO₄-5H₂O 20-40 μM, CuCl₂-2H₂O 4-8 μM, H₃BO₃ 6-10 μM, Na₂MoO₄-2H₂O 6-10 μM, ZnSO₄-7H₂O 4-8 μM, FeSO₄-7H₂O 75-125 μM, CaCl₂-2H₂O 40-100 μM, AlCl₃-6H₂O 2-6 μM and NiCl₂-6H₂O 6-10 μM. In certain embodiments, a metal mix may be prepared as a dry mix or a concentrate. A dry mix or concentrate will have proportional amounts of each metal such that upon reconstitution in water or upon addition to media, the appropriate concentration of each metal is obtained. In certain embodiments, it may be desirable to adjust the pH with an acid or base when reconstituting the metal mix to facilitate solvation of the metals. In an exemplary embodiment, a concentrated metal solution is made up in water supplemented with 0.1% concentrated HCl. Concentrates are often referred to in terms of the amount of dilution that is recommended to achieve the intended metal concentrations. For example, a 100× metal concentrate has a concentration of each metal that is 100 times higher than the concentration that is intended for use in cell culture. By mixing 1 mL of 100× metal concentrate with 99 mL of medium, the desired metal concentration is obtained.

In certain embodiments, a medium to be employed in a method disclosed herein comprises one or more vitamins in addition to whatever vitamins may be provided by any complex biological extracts. Exemplary vitamins that may be included in a medium are: p-aminobenzoic acid, L-ascorbic acid, biotin, D-pantothenate, choline, folic acid, myo-inositol, nicotinamide, pyridoxine, riboflavin, thiamine, vitamin A, vitamin B12 and vitamin D. Most vitamins are weak acids or bases (or may have both acidic and basic moieties) and may be supplied in the acidic or basic form or with a suitable counterion. For example, choline may be supplied as a salt with an anion such as chloride, pantothenate may be supplied as D-calcium pantothenate, and pyridoxine may be supplied as pyridoxine-HCl. Exemplary media comprise one, two, three, four, five, six, seven, eight, nine, ten, eleven, twelve, thirteen, or fourteen of the preceding vitamins. Recommended concentration ranges for each of the preceding vitamins are as follows (mg/L final concentration in media): p-aminobenzoic acid 0.005-0.05, L-ascorbic acid 0.5-5, biotin 0.003-0.03, D-calcium pantothenate 0.03-0.3, choline chloride 0.03-0.3, folic acid 0.1-1.0, myo-inositol 30-300, nicotinamide 0.3-3.0, pyridoxine-HCl 0.3-3.0, riboflavin 0.08-0.8, thiamine-HCl 0.3-3.0, vitamin A 0.003-0.03, vitamin B12 0.005-0.05, and vitamin D 0.003-0.03. Suitable concentrated mixtures of vitamins are commercially available, such as Kao and Michayluk vitamin solution 100× (K3129, Sigma-Aldrich, St. Louis, Mo.).

In certain embodiments, a medium to be employed in a method disclosed herein is a minimal medium comprising one or more amino acid supplements. Exemplary amino acids that may be included in a medium are free base L-amino acids including: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and various analogs and derivatives thereof. Exemplary media may comprise one, two, three, four, five, six, seven, eight, nine, ten, eleven, twelve, thirteen, fourteen, fifteen, sixteen, seventeen, eighteen, nineteen, or twenty of the preceding amino acids. Recommended concentration ranges for each of the preceding amino acids are as follows (final concentration in media): alanine 400-500 μM, arginine 150-250 μM, asparagine 250-350 μM, aspartic acid 250-350 μM, cysteine 280-380 μM, glutamic acid 220-320 μM, glutamine 220-320 μM, glycine 480-580 μM, histidine 140-240 μM, isoleucine 250-350 μM, leucine 250-350 μM, lysine 170-270 μM, methionine 150-250 μM, phenylalanine 200-300 μM, proline 300-400 μM, serine 330-430 μM, threonine 280-380 μM, tryptophan 150-250 μM, tyrosine 170-270 μM, and valine 290-390 μM. Based on the teachings herein, one of skill in the art will be able to determine an appropriate amino acid supplement for growth of a given host cell and/or labeling of a polypeptide with a given label. In certain embodiments, an amino acid mix comprising one or more of the above amino acids may be prepared as a dry mix or concentrate. Amino acids may be purchased, for example, from Sigma (L-Amino Acids Free Base, Sigma, Catalogue Number LAA21).

In certain embodiments, a medium to be employed in a method disclosed herein comprises a label that may be taken up by the cells and incorporated into the protein. In certain embodiments, suitable labels include labels that facilitate the functional and/or structural characterization of a polypeptide, including, for example, radioisotopes, fluorescent labels, chemiluminescent groups, heavy atoms, and seleno-L-methionine. In one embodiment, a medium may comprise a label that facilitates structural characterization of a polypeptide by NMR, including, for example, an isotopic label, such as potassium-40 (⁴⁰K), carbon-14 (¹⁴C), tritium (³H), sulphur-35 (³⁵S), phosphorus-32 (³²P), technetium-99m (^{99m}Tc), thallium-201 (²⁰¹Tl), gallium-67 (⁶⁷Ga), indium-111 (¹¹¹In), iodine-123 (¹²³I), iodine-131 (¹³¹I), yttrium-90 (⁹⁰Y), samarium-153 (¹⁵³Sm), rhenium-186 (¹⁶⁶Re), rhenium-188 (¹⁸⁸Re), dysprosium-165 (¹⁶⁵Dy), holmium-166 (¹⁶⁶ Ho), hydrogen-1 (¹H), hydrogen-2 (²H), hydrogen-3 (³H), phosphorous-31 (³¹P), sodium-23 (²³Na), nitrogen-14 (¹⁴N), nitrogen-15 (¹⁵N), carbon-13 (¹³C) or fluorine-19 (¹⁹F). In another embodiment, a medium may comprise a label that facilitates structural characterization of a polypeptide by x-ray crystallography, for example, a heavy atom label, such as cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium, or uranium. In an exemplary embodiment, the medium comprises seleno-L-methionine at a final concentration of 100-300 μM. Seleno-L-methionine is commercially available (e.g., Sigma; Cat. No. S3132). When producing a seleno-L-methionine labeled polypeptide using the methods disclosed herein, it may be preferable to use a minimal medium substituted with all of the L-amino acids listed above but substituting seleno-L-methionine for the standard form of L-methionine.

Exemplary rich media that may be employed in generating a high density cell culture include Luria broth (“LB”), Terrific broth (“TB”), Super broth and Z broth (all available from Bio-Whittaker, Inc., Walkersville, Md.). Surprisingly, certain media mixtures are particularly effective for generating a high density cell culture. In certain embodiments, the invention relates to an exemplary medium comprising: 10-14 g/L tryptone, 20-30 g/L of yeast extract, buffering salts at an initial pH of between 6 and 8, and one or more metals such as cobalt, manganese, copper, boron, molybdenum, zinc, iron, calcium, aluminum and nickel. Optionally, the medium further comprises 0.5-5% glycerol, and optionally about 1.5% glycerol. Optionally the buffer salts comprise phosphates, such as potassium or sodium phosphate (e.g. 100-200 mM phosphate concentration). Optionally, the medium comprises one or more of the following metals at the recommended concentration ranges: CoCl₂-6H₂O 4-8 μM, MnSO₄-5H₂O 20-40 μM, CuCl₂-2H₂O 4-8 μM, H₃BO₃ 6-10 μM, Na₂MoO₄-2H₂O 6-10 μM, ZnSO₄-7H₂O 5-9 μM, FeSO₄-7H₂O 75-125 μM, CaCl₂-2H₂O 40-100 μM, AlCl₃-6H₂O 2-6 μM and NiCl₂-6H₂O 6-10 μM. Optionally the medium comprises a magnesium source, such as MgSO₄, so as to provide a magnesium concentration of 0.5-2 mM. Optionally the medium comprises one or more of the following vitamins at the recommended concentration ranges (mg/L final concentration in media): p-aminobenzoic acid 0.005-0.05, L-ascorbic acid 0.5-5, biotin 0.003-0.03, D-calcium pantothenate 0.03-0.3, choline chloride 0.03-0.3, folic acid 0.1-1.0, myo-inositol 30-300, nicotinamide 0.3-3.0, pyridoxine-HCl 0.3-3.0, riboflavin 0.08-0.8, thiamine-HCl 0.3-3.0, vitamin A 0.003-0.03, vitamin B12 0.005-0.05, and vitamin D 0.003-0.03.

Exemplary minimal media that may be employed in generating a high density cell culture include Bio-express 1000 (CIL), Bio-express min (Cambridge Isotope Laboratories, Inc., Andover, Mass.), Minimal Medium M9, Silantes (VLI Research, Inc., Malvern, Pa.), and Martek 9 (Martek Biosciences Corp., Columbia, Md.). In certain embodiments, the invention relates to an exemplary medium for producing seleno-L-methionine labeled polypeptides comprising: 0.04-4% glucose, NH₄Cl at a concentration of 20-60 mM, KH₂PO₄ at a concentration of 20-60 mM, Na₂HPO₄ at a concentration of 75-115 mM, Na₂HPO₄-7H₂O at a concentration of 75-115 mM, MgSO₄ at a concentration of 0.5-4 mM, FeSO₄ at a concentration of 70-110 mM, CaCl₂ at a concentration of 80-120 μM, one or more amino acids but not methionine, vitamins, and seleno-L-methionine at a concentration of 100-300 μM.

Media described herein may be prepared as a dry mix or a concentrate. A dry mix will generally contain less than about 20% H₂O by weight, and may be in a powder or other solid or semisolid form. Some components, such as glycerol, tend to be liquids even in the absence of water. Media may also be prepared as a concentrate, such as a 100×, 20×, 10×, 5×, or 2× concentrate.

Other components may be incorporated as needed. For example, media for growing cultures of bacteria carrying a plasmid having an antibiotic resistance cassette may include one or more antibiotics, such as ampicillin, carbenicillin, chloramphenicol, streptamycin, neomycin, gentamycin, kanamycin, phleomycin, bleomycin, nalidixic acid, tetracycline, etc.

4. Cell Culture Products

High density cell cultures may be used for any of the various purposes that such cultures are generally used for. For example, a high cell density cell culture may be used to obtain one or more desired cell products, such as metabolites, proteins or nucleic acids. Exemplary metabolites include amino acids, lipids, vitamins, sugars, antibiotics, polysaccharides, polyhydroxybutyrates, etc. Exemplary proteins include recombinant and non-recombinant proteins, either of which may be used, for example, for therapeutic, industrial or research purposes. Exemplary nucleic acids include genomic DNA, total RNA, mRNA, viral nucleic acids, plasmids, cosmids and artificial chromosomes. In exemplary embodiments, the compositions and methods of the invention are used in conjunction with high-throughput methods for handling multiple samples simultaneously. Various robotic and computer assisted instruments may be used for processing and handling large numbers of samples as appropriate. In certain embodiments, the methods and compositions of the invention may be incorporated into an auto-purification pipeline for the production of large numbers of purified polypeptides in parallel. Other uses for high density cell cultures will, in view of this specification, be apparent to one of skill in the art.

A cell culture for use in producing a recombinant polypeptide will generally comprise cells that carry a recombinant nucleic acid expression construct for expressing a nucleic acid encoding the desired polypeptide. In certain embodiments, the expression construct is an exogenously regulated expression construct such that production of the polypeptide may be influenced by addition or omission of one or more external factors, such as inducers. Exemplary exogenously regulated expression constructs and inducers include P_{lac} promoters (IPTG-inducible), P_{xyl} promoters (xylose-inducible) and tet promoters (tetracycline inducible). In certain instances, it is desirable to induce polypeptide production only at a later stage of growth, such as when the cell culture reaches an OD₆₀₀ of between 4 and 10. Optionally, cells that produce a particular protein at high levels may be induced at a higher OD₆₀₀, such as between 6 and 10, while cells that produce a protein at low levels may be induced at a lower OD₆₀₀, such as between 4 and 8. Cells may be cultured at a constant temperature, or optionally, the temperature may be different before and after protein expression is induced. For example, a cell culture may be substantially maintained at greater than 20° C. (optionally greater than 15° C., 25° C., or 30° C.) prior to induction and substantially maintained at lower than 20° C. (optionally lower than 15° C., 25° C., or 30° C.) after induction.

Recombinant nucleic acid expression constructs may be generated in any of the various ways known in the art. For example, a nucleic acid to be expressed may be amplified by polymerase chain reaction (PCR) and directionally cloned into the polylinker region of an expression vector such as: pET28 (Novagen), pET15 (Novagen) or pGEX (Pharmacia/LKB Biotechnology). After the ligation reaction, the DNA may be transformed into competent cells, such as E. coli cells (e.g. strains XL1-Blue (Stratagene) or DH5α (Invitrogen)) via heat shock or electroporation as described in Sambrook, et al., Molecular Cloning: A Laboratory Manual, 2nd Ed., Cold Spring Harbor Laboratory Press, Cold Spring Harbor, N.Y. (1989). In an exemplary embodiment, expression vectors containing the bacteriophage T7 promoter for RNA polymerase are used in conjunction with an E. coli strain that produces T7 RNA polymerase upon induction with isopropyl-β-D-thiogalactoside (IPTG). In certain embodiments, cloning vectors that add a Glutathione S-transferase (GST) tag and/or a polyhistidine (6×His) tag at the N- and/or C-terminus of the recombinant protein may be used. A cleavage site for the thrombin or Tev (Invitrogen) enzymes may optionally be included between the recombinant protein and the N- or C-terminal tags to facilitate their removal. Transformants may be selected using an appropriate antibiotic (e.g. Ampicillin or Kanamycin) and identified using PCR, or another method, to analyze their DNA. The polynucleotide sequence cloned into the expression construct may be isolated, for example, using a modified alkaline lysis method (Birnboim, H. C., and Doly, J. (1979). The sequence of the clone may be verified by standard polynucleotide sequencing methods.

An expression construct may be introduced into a host cell by an appropriate method such as electroporation, liposome-mediated transfection, calcium chloride transformation, viral infection, etc. For production of proteins in bacteria, many suitable expression strains are available, such as, for example, the BL21-Gold DE3™ strain. Optionally, the BL-21-Gold DE3™ strain may be supplemented with a plasmid called ‘Magic’ which directs expression of three tRNAs rarely employed in the host cell (agg, aga, and ata) and serves to augment the expression of the recombinant protein in the host cell. The expression construct may also be transformed into BL21-Gold-DE3 Codon Plus™ (Stratagene) which contains genes encoding for a different set of rarely used tRNAs (cgg, cga, and cta). As a further exemplary option, the expression construct may be transformed into BL21 STAR™ E. coli (Invitrogen) cells which has an RNase deficiency that reduces degradation of recombinant mRNA transcript and therefore increases the protein yield. The recombinant protein may be assayed for positive overexpression in the host and the presence of the protein in the cytoplasmic (water soluble) region of the cell.

In certain embodiments, the coding sequences for a polypeptide of interest may be a fusion gene comprising a heterologous polypeptide that increases solubility and/or facilitates detection, identification, isolation, and functional and/or structural characterization. In an exemplary embodiment, a fusion gene coding for a poly-(His) tag fused to the N- and/or C-terminus of a polypeptide of interest may be used to purify the expressed protein by affinity chromatography with an Ni²⁺ metal resin. The tag may optionally be separated from the polypeptide by treatment with sequence-specific endoprotease (e.g., see Hochuli et al., (1987) J. Chromatography 411: 177; and Janknecht et al., PNAS USA 88:8972). Techniques for making fusion genes are well known. Joining of various DNA fragments coding for different polypeptide sequences may be performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Alternatively, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992).

The methods disclosed herein may be employed with any appropriate preparative technique. For example, a host cell transfected with an expression vector encoding a polypeptide may be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide may be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of a polypeptide, removal of insoluble materials (in the case of a soluble protein) or removal of soluble materials (in the case of an insoluble protein), ammonium sulfate precipitation, hydrophobic interaction chromatography, reverse phase chromatography, affinity chromatography, size exclusion chromatography, and various combinations thereof.

Polypeptides produced using the methods disclosed herein may be used for any application that employs polypeptides, and the downstream use will generally dictate the additional preparative or experimental steps to be carried out. In certain embodiments, the compositions and methods described herein may be used for structural and/or functional characterization of a polypeptide, including, for example, affinity chromatography, mass spectrometry, NMR, x-ray crystallography, antibody production, screening assays to identify modulators of biological activity, etc. The invention further provides methods and compositions for producing polypeptides that are modified to facilitate their characterization, including polypeptides labeled with isotopic or heavy atoms and fusion proteins.

In exemplary embodiments, polypeptides prepared in accordance with the compositions and methods described herein may be used for determining three dimensional structural information of a polypeptide using mass spectroscopy, NMR, and X-ray crystallography, and various combinations thereof. In an exemplary embodiment, a seleno-L-methionine labeled polypeptide prepared in accordance with the compositions and methods disclosed herein may be used for x-ray crystallographic analysis of the three dimensional structure of a polypeptide.

While it is anticipated that any polypeptide may be prepared using the methods and compositions disclosed herein, exemplary polypeptides include, for example, kinases, proteases, phosphatases, P450s, conjugation enzymes, ATPases, GTPase, nucleotide binding proteins, DNA processing enzymes, helicases, polymerases, RNA polymerases, DNA polymerases, GPCRs, intracellular receptors, metabolic enzymes, nuclear receptors, channels, phosphodiesterases, essential bacterial proteins, Ca²⁺ binding proteins, bacterial proteins, non-membrane bacterial proteins, human proteins that bind viral proteins, viral proteins, and nonmembrane viral proteins. In exemplary embodiments, the polypeptides which are used in accordance with the methods of the invention are bacterial proteins derived from Eschericia coli, Helicobacter pylori, Pseudomonas aeruginosa, Chlamydia trachomatis, Haemophilus influenzae, Neisseria meningitidis, Rickettsia prowazekii, Borrelia burgdorferi, Bacillus subtilis, Staphylococcus aureus, Staphylococcus epidermis, Streptococcus pneumoniae, Mycoplasma genitalium, Salmonella typhimurium, or Enterococcus faecalis.

In certain embodiments, NMR may be used to determine structure information of a polypeptide prepared in accordance with the compositions and methods described herein. In particular, NMR may be used, for example, to determine the three dimensional structure, the conformational state, the aggregation level, the state of protein folding/unfolding or the dynamic properties of a polypeptide. Changes in these properties due to interaction with other molecules, including both small molecules (drugs, co-factors, etc.) and macromolecules (e.g., polynucleotides, polypeptides, etc.), can also be monitored using NMR.

Polypeptides in aqueous solution usually populate an ensemble of 3-dimensional (3D) structures which can be determined by NMR. The 2-dimensional ¹H-¹⁵N HSQC (Heteronuclear Single Quantum Correlation) spectrum provides a diagnostic fingerprint of conformational state, aggregation level, state of protein folding, and dynamic properties of a polypeptide (Yee et al, PNAS 99, 1825-30 (2002)). When the polypeptide is a stable globular protein or domain of a protein, then the ensemble of solution structures is one of very closely related conformations. In this case one peak is expected for each non-proline residue with a dispersion of resonance frequencies with roughly equal intensity. Additional pairs of peaks from side-chain NH₂ groups are also often observed, and correspond to approximately the number of Gln and Asn residues in the protein. This type of HSQC spectra usually indicates that the protein is amenable to structure determination by NMR methods.

In other embodiments, x-ray crystallography may be used to determine structure information of a polypeptide prepared in accordance with the compositions and methods described herein. In particular, x-ray diffraction of a crystallized form of a polypeptide can be used, for example, to determine the three dimensional structure of a polypeptide or to determine the space group of the crystal of the polypeptide. Exemplary methods for obtaining the three dimensional structure of a crystalline form of a molecule or complex will be apparent to those skilled in the art based on the teachings herein (see Ducruix and Geige 1992, IRL Press, Oxford, England).

X-ray crystallography techniques generally require that the protein molecules be available in the form of a crystal. Crystals may be grown from a solution containing a purified polypeptide, or a fragment thereof (e.g., a stable domain), by a variety of conventional processes. These processes include, for example, batch, liquid, bridge, dialysis, vapour diffusion (e.g., hanging drop or sitting drop methods). See for example, McPherson, 1982 John Wiley, New York; McPherson, 1990, Eur. J. Biochem. 189: 1-23; Webber. 1991, Adv. Protein Chem. 41:1-36. In certain embodiments, native crystals of a polypeptide may be grown by adding precipitants to a concentrated solution of the polypeptide. The precipitants are added at a concentration just below that necessary to precipitate the protein. Water may be removed by controlled evaporation to produce precipitating conditions, which are maintained until crystal growth ceases. The formation of crystals is dependent on a number of different parameters, including pH, temperature, protein concentration, the nature of the solvent and precipitant, as well as the presence of added ions or ligands to the protein. In addition, the sequence of the polypeptide being crystallized will have a significant affect on the success of obtaining crystals. Many routine crystallization experiments may be needed to screen all these parameters for the few combinations that might give crystal suitable for x-ray diffraction analysis (See, for example, Jancarik, J & Kim, S. H., J. Appl. Cryst. 1991 24: 409-411). Crystallization robots may automate and speed up the work of reproducibly setting up large number of crystallization experiments. Once some suitable set of conditions for growing a crystal are found, variations of the conditions may be systematically screened in order to find the set of conditions which allows the growth of sufficiently large, single, well ordered crystals. In certain instances, a polypeptide may be co-crystallized with a compound that stabilizes the polypeptide.

In certain embodiments, it may be useful to determine the three dimensional structure of a crystallized polypeptide in the presence of another molecule, such as another polypeptide, nucleic acid or small molecule. In such embodiments, a polypeptide may be co-crystallized with another molecule in order to provide a crystal suitable for determining the structure of the complex. Alternatively, a crystal of the polypeptide may be soaked in a solution containing the other molecule in order to form co-crystals by diffusion of the other molecule into the crystal of the polypeptide. In exemplary embodiments, the structure of the polypeptide obtained in the presence and absence of another molecule may be compared to determine structural information about the polypeptide and aid in identification of druggable regions.

A number of methods are available to produce suitable radiation for X-ray diffraction. For example, x-ray beams may be produced by synchrotron rings where electrons (or positrons) are accelerated through an electromagnetic field while traveling at close to the speed of light. Because the admitted wavelength may also be controlled, synchrotrons may be used as a tunable x-ray source (Hendrickson W. A., Trends Biochem Sci 2000 December; 25(12):637-43). For less conventional Laue diffraction studies, polychromatic x-rays covering a broad wavelength window may be used to observe many diffraction intensities simultaneously (Stoddard, B. L., Curr. Opin. Struct Biol 1998 October; 8(5):612-8). Neutrons may also be used for solving protein crystal structures (Gutberlet T, Heinemann U & Steiner M., Acta Crystallogr D 2001;57: 349-54).

In other embodiments, mass spectrometry may be used to determine structure information of a polypeptide prepared in accordance with the compositions and methods described herein. In particular, mass spectrometry can be used, for example, to determine the amino acid sequence, to obtain a peptide map, to identify post-translational modifications (e.g., phosphorylation, etc.) of a polypeptide, or to identifying regions of the polypeptide that interact with other molecules, including other polypeptides, nucleic acids and small molecules.

In certain embodiments, a polypeptide may be subjected to limited proteolysis prior to analysis by mass spectrometry. Limited proteolysis of a polypeptide may be used to identify and/or isolate stable domains of a protein that are suitable for structural characterization using NMR analysis or x-ray crystallography. Limited proteolysis of a polypeptide may be performed by incubating a protein with at least one concentration of a proteolytic enzyme for an amount of time suitable to produce proteolytic cleavage of the protein of interest. In certain embodiments, digestion of the polypeptide may be carried out by incubation with two or more proteolytic enzymes, at two or more concentrations of enzyme, and/or for varying amounts of time. Such reactions may be carried out in solution or by exposing the polypeptide to an immobilized proteolytic enzyme to facilitate isolation of the polypeptide fragments from the digestion mixture. The digestion products may be analyzed and/or isolated using electrophoretic or chromatographic techniques. Proteolytically stable fragments resulting from the enzymatic digestion may be identified based on the mass of the peptide as determined by mass spectrometry.

Exemplification

The invention now being generally described, it will be more readily understood by reference to the following examples, which are included merely for purposes of illustration of certain aspects and embodiments of the present invention, and are not intended to limit the invention.

### EXAMPLE 1

### High Density Cell Culture for Protein Production

A starter culture is prepared in a 300 mL Tunair flask (Shelton Scientific) by adding 20 mL of medium having 47.6 g/L of Terrific Broth and 1.5% glycerol in dH₂O followed by autoclaving for 30 minutes at 121° C. and 15 psi. When the broth cools to room temperature, the medium is supplemented with 6.3 μM CoCl₂-6H₂O, 33.2 μM MnSO₄-5H₂O, 5.9 μM CuCl₂-2H₂O, 8.1 μM H₃BO₃, 8.3 μM Na₂MoO₄-2H₂O, 7 μM ZnSO₄-7H₂O, 108 μM FeSO₄-7H₂O, 68 μM CaCl₂-2H₂O, 4.1 μM AlCl₃-6H₂O, 8.4 μM NiCl₂-6H₂O, 1 mM MgSO₄, 0.5% v/v of Kao and Michayluk vitamins mix (Sigma; Cat. No. K3129), 25 μg/mL Carbenicillin, and 50 μg/mL Kanamycin. The medium is then inoculated with several colonies of E. coli BL21(DE3) Gold™ cells (Stratagene) freshly transformed with an expression construct harboring a gene of interest. The culture is incubated at 37° C. and 260 rpm for about 3 hours and then transferred to a 2.5L Tunair Flask containing 1L of the above media. The 1L culture is then incubated at 37° C. with shaking at 230-250 rpm on an orbital shaker having a 1 inch orbital diameter. When the culture reaches an OD₆₀₀ of 3-6 (generally lower for cells expressing proteins that express poorly and higher for cells expressing proteins that express well) it is induced with 0.5 mM IPTG. The induced culture is then incubated at 15° C. with shaking at 230-250 rpm or faster for about 6-15 hours. The cells are harvested by centrifugation and resuspended in an appropriate buffer.

For a 100 mL growth, prepare a 2 mL starter culture with the above media in a sterile 10 mL test tube and shake at 37° C. and 270-300 rpm for about 3 hours with the rack of the orbital shaker slightly tilted to increase the aeration surface area. Transfer the 2 mL starter culture to the 100 mL main culture and proceed as described above for the 1 L culture, but shake the cultures at 270-300 rpm.

### EXAMPLE 2

### High Density Cell Culture Production of Seleno-L-Methionine Labeled Polypeptides

A starter culture is prepared in a 300 mL Tunair flask (Shelton Scientific) by adding 50 mL of sterile medium having 10% 10XM9 (37.4 mM NH₄Cl (Sigma; Cat. No. A4514), 44 mM KH₂PO₄ (Bioshop, Ontario, Canada; Cat. No. PPM 302), 96 mM Na₂HPO₄ (Sigma; Cat. No. S2429256), and 96 mM Na₂HPO₄-7H₂O (Sigma; Cat. No. S9390) final concentration), 450 μM alanine, 190 μM arginine, 302 μM asparagine, 300 μM aspartic acid, 330 μM cysteine, 272 μM glutamic acid, 274 μM glutamine, 533 μM glycine, 191 μM histidine, 305 μM isoleucine, 305 μM leucine, 220 μM lysine, 242 μM phenylalanine, 348 μM proline, 380 μM serine, 336 μM threonine, 196 μM tryptophan, 220 μM tyrosine, and 342 μM valine, 204 μM Seleno-L-Methionine (Sigma; Cat. No. S3132), 0.5% v/v of Kao and Michayluk vitamins mix (Sigma; Cat. No. K3129), 2 mM MgSO₄ (Sigma; Cat. No. M7774), 90 μM FeSO₄-7H₂O (Sigma; Cat. No. F8633), 0.4% glucose (Sigma; Cat. No. G-5400), 100 μM CaCl₂ (Bioshop, Ontario, Canada; Cat. No. CCL 302), 50 μg/mL Ampicillin, and 50 μg/mL Kanamycin in dH₂O. The medium is then inoculated with several colonies of E. coli B834 cells (Novagen) freshly transformed with an expression construct harboring a gene of interest. The culture is then incubated at 37° C. and 200 rpm until it reaches an OD₆₀₀ of ˜1 (approximately 3 hours to overnight) and is then transferred to a 2.5L Tunair Flask containing 1L of the above media. The 1L culture is incubated at 37° C. with shaking at 200 rpm until the culture reaches an OD₆₀₀ of 0.6-0.8 and is then induced with 0.5 mM IPTG. The induced culture is incubated overnight at 15° C. with shaking at 200 rpm. The cells are harvested by centrifugation and resuspended in an appropriate buffer.

Equivalents

The present invention provides among other things novel methods and compositions for growth of cell cultures and protein production. While specific embodiments of the subject invention have been discussed, the above specification is illustrative and not restrictive. Many variations of the invention will become apparent to those skilled in the art upon review of this specification. The appended claims are not intended to claim all such embodiments and variations, and the fill scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: WO 00/45168, WO 00/79238, WO 00/77712, EP 1047108, EP 1047107, WO 00/72004, WO 00/73787, WO00/67017, WO 00/48004, WO 00/45168, WO 00/45164, U.S. Ser. No. 09/720,272; U.S. S. No. 60/399,873, filed Jul. 31, 2002; PCT/CA99/00640; U.S. Pat. Nos. 6,254,833; 6,232,114; 6,229,603; 6,221,612; 6,214,563; 6,200,762; 6,171,780; 6,143,492; 6,124,128; 6,107,477; D428,157; 6,063,338; 6,004,808; 5,985,214; 5,981,200; 5,928,888; 5,910,287; 6,248,550; 6,232,114; 6,229,603; 6,221,612; 6,214,563; 6,200,762; 6,197,928; 6,180,411; 6,171,780; 6,150,176; 6,140,132; 6,124,128; 6,107,066; 6,077,707; 6,066,476; 6,063,338; 6,054,321; 6,054,271; 6,046,925; 6,031,094; 6,008,378; 5,998,204; 5,981,200; 5,955,604; 5,955,453; 5,948,906; 5,932,474; 5,925,558; 5,912,137; 5,910,287; 5,866,548; 5,834,436; 5,777,079; 5,741,657; 5,693,521; 5,661,035; 5,625,048; 5,602,258; 5,552,555; 5,439,797; 5,374,710; 5,296,703; 5,283,433; 5,141,627; 5,134,232; 5,049,673; 4,806,604; 4,689,432; 4,603,209; 6,217,873; 6,174,530; 6,168,784; 6,271,037; 6,228,654; 6,184,344; 6,040,133; 5,910,437; 5,891,993; 5,854,389; 5,792,664; 6,248,558; 6,341,256; 5,854,922; 5,866,343; 4,665,035; Sandhu et al., Archives Biochem. Biophys. 309(1): 168-177 (1994); and Smith et al., Biochem. J. 331: 783-792 (1998).

What is claimed:

1. A method of generating a high density cell culture, the method comprising:
a) placing cells and a medium in a simple culture vessel, thereby generating a cell culture, the medium being suitable for growth of a high density cell culture; and
b) operating the simple culture vessel to provide an oxygen transfer rate suitable for growth of a high density cell culture, thereby generating a high density cell culture, the high density cell culture having an optical density at a wavelength of 600 nm (OD₆₀₀) of 4 or greater.

2. The method of claim 1, wherein the oxygen transfer rate is greater than 2.0 millimoles O₂/liter/minute.

3. The method of claim 1, wherein the cells are bacterial cells.

4. The method of claim 3, wherein a substantial portion of the bacterial cells comprise an exogenously regulated expression construct.

5. The method of claim 4, further comprising contacting the cell culture with an inducer.

6. The method of claim 4, wherein the exogenously regulated expression construct comprises a lacI binding site operably linked to an expressible nucleic acid, and wherein the inducer is IPTG.

7. The method of claim 5, wherein the cell culture is contacted with the inducer when the culture has an OD₆₀₀ of 1 or greater.

8. The method of claim 5, wherein the cell culture is substantially maintained at a temperature higher than 25° C. prior to contacting the cell culture with the inducer and the cell culture is substantially maintained at a temperature lower than 25° C. after contacting the cell culture with the inducer.

9. The method of claim 1, wherein the simple culture vessel is shaken on an orbital shaker table with an orbital diameter and shaking rate (revolutions per minute) suitable to provide an oxygen transfer rate greater than 2.0 millimoles O₂/liter/minute.

10. The method of claim 9, wherein the orbital shaker table has an orbital diameter of at least about one inch and the shaking rate is at least about 200 revolutions per minute.

11. The method of claim 1, wherein the simple culture vessel is shaken at greater than 250 revolutions per minute.

12. The method of claim 11, wherein the cell culture has a volume of less than 200 milliliters.

13. The method of claim 1, wherein the optical density at a wavelength of 600 nm is 10 or greater.

14. The method of claim 13, wherein the cell culture has a volume of between 500 and 2000 milliliters.

15. The method of claim 1, wherein the optical density at a wavelength of 600 nm is 20 or greater.

16. The method of claim 15, wherein the cell culture has a volume of less than 200 milliliters.

17. The method of claim 1, wherein the medium comprises:
i) a carbon source selected from the group consisting of glycerol and glucose;
ii) a complex organic material selected from the group consisting of tryptone, yeast extract, hydrolyzed casein and beef broth; and
iii) a magnesium source.

18. The method of claim 17, the medium further comprising:
iv) two or more metals selected from the group consisting of cobalt, manganese, copper, boron, molybdenum, zinc, iron, calcium, aluminum and nickel.

19. The method of claim 1, wherein the medium comprises:
i) tryptone at a concentration of 10-14 grams/L;
ii) yeast extract at a concentration of 20-30 grams/L;
iii) buffering salts at an initial pH of between 6 and 8;
iv) magnesium sulfate at a concentration of between 0.5 and 2 mM;
v) 0.5-5% glycerol; and
vi) a metal mixture comprising cobalt, manganese, copper, boron, molybdenum, zinc, iron, calcium, aluminum and nickel.

20. The method of claim 19, wherein the buffering salts comprise potassium and phosphate, the phosphate at a concentration of between 100 and 200 mM.

21. The method of claim 19, wherein the metal mixture comprises: CoCl₂-6H₂O (4-8 μM), MnSO₄-5H₂O (20-40 μM), CuCl₂-2H₂O (4-8 μM), H₃BO₃ (6-10 μM), Na₂MoO₄-2H₂O (6-10 μM), ZnSO₄-7H₂O (4-8 μM), FeSO₄-7H₂O (75-125 μM), CaCl₂-2H₂O (40-100 μM), AlCl₃-6H₂O (2-6 μM), and NiCl₂-6H₂O (6-10 μM).

22. The method of claim 1, further comprising obtaining a partially purified polypeptide composition from the high density cell culture.

23. The method of claim 1, further comprising obtaining a purified polypeptide from the high density cell culture.

24. The method of claim 23, the method further comprising determining an NMR structure of the polypeptide.

25. The method of claim 23, the method further comprising determining an X-ray crystal structure of the polypeptide.

26. The method of claim 23, the method further comprising determining a druggable region of the polypeptide.

27. The method of claim 1, wherein the simple culture vessel is a Tunac-type flask.

28. The method of claim 1, wherein the simple culture vessel is a 96-well plate.

29. A high density cell culture produced according to the method of claim 1.

30. A partially purified polypeptide composition produced according to the method of claim 22.

31. A purified polypeptide composition produced according to the method of claim 23.

32. The purified polypeptide composition of claim 31, wherein the polypeptide composition comprises a polypeptide selected from the group consisting of: a therapeutically useful polypeptide, an industrially useful polypeptide and a polypeptide useful for research purposes.

33. The method of claim 1, further comprising contacting the cell culture with a label that may be incorporated into a polypeptide during growth of the high density cell culture.

34. The method of claim 33, wherein the label is seleno-L-methionine.

35. The method of claim 33, wherein the label is an isotopic label selected from the group consisting of potassium-40 (⁴⁰K), carbon-14 (¹⁴C), tritium (³H), sulphur-35 (³⁵S), phosphorus-32 (³²P), technetium-99m (^{99m}Tc), thallium-201 (²⁰¹Tl), gallium-67 (⁶⁷Ga), indium-111 (¹¹¹In), iodine-123 (¹²³I), iodine-131 (¹³¹I), yttrium-90 (⁹⁰Y), samarium-153 (¹⁵³Sm), rhenium-186 (¹⁸⁶Re), rhenium-188 (¹⁸⁸Re), dysprosium-165 (¹⁶⁵Dy), holmium-166 (¹⁶⁶Ho), hydrogen-1 (¹H), hydrogen-2 (²H), hydrogen-3 (³H), phosphorous-31 (³¹P), sodium-23 (²³Na), nitrogen-14 (¹⁴N), nitrogen-15 (¹⁵N), carbon-13 (¹³C) and fluorine-19 (¹⁹F).

36. The method of claim 33, wherein the label is a heavy atom label selected from the group consisting of cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium.

37. The method of claim 1, wherein the medium comprises:
i) a carbon source selected from the group consisting of glycerol and glucose;
ii) a basal nutrient source; and
iii) a label selected from the group consisting of an isotopic label, a heavy atom label, and seleno-L-methionine.

38. The method of claim 37, the medium further comprising one or more amino acids.

39. The method of claim 37, wherein the medium comprises: 0.04-4% glucose, NH₄Cl at a concentration of 20-60 mM, KH₂PO₄ at a concentration of 20-60 mM, Na₂HPO₄ at a concentration of 75-115 mM, Na₂HPO₄-7H₂O at a concentration of 75-115 mM, MgSO₄ at a concentration of 0.5-4 mM, FeSO₄ at a concentration of 70-110 mM, CaCl₂ at a concentration of 80-120 μM, one or more amino acids but not methionine, vitamins, and seleno-L-methionine at a concentration of 100-300 μM.

40. A method of culturing cells, the method comprising:
a) placing cells and a medium in a simple culture vessel, thereby generating a cell culture, the medium comprising:
i) tryptone at a concentration of 10-14 grams/L;
ii) yeast extract at a concentration of 20-30 grams/L;
iii) buffering salts at a pH of between 6 and 8; and
iv) MgSO₄ at a concentration of between 0.5 and 2 mM; and
b) operating the simple culture vessel to provide an oxygen transfer rate greater than 2.0 millimoles O₂/liter/min.

41. The method of claim 40, wherein the medium further comprises:
v) 0.5-5% glycerol;
vi) a metal mixture comprising: cobalt, manganese, copper, boron, molybdenum, zinc, iron, calcium, aluminum and nickel; and
vii) vitamins.

42. The method of claim 41, wherein the metal mixture comprises: CoCl₂-6H₂O (4-8 μM), MnSO₄-5H₂O (20-40 μM), CuCl₂-2H₂O (4-8 μM), H₃BO₃ (6-10 μM), Na₂MoO₄-2H₂O (6-10 μM), ZnSO₄-7H₂O (4-8 μM), FeSO₄-7H₂O (75-125 μM), CaCl₂-2H₂O (40-100 μM), AlCl₃-6H₂O (2-6 μM), and NiCl₂-6H₂O (6-10 μM).

43. The method of claim 40, wherein the simple culture vessel is a Tunac-type flask.

44. A method for producing a labeled polypeptide, the method comprising:
a) placing cells and a medium in a simple culture vessel, thereby generating a cell culture, the medium comprising:
i) a carbon source selected from the group consisting of glycerol and glucose;
ii) a basal nutrient source; and
iii) a label selected from the group consisting of an isotopic label, a heavy atom label, and seleno-L-methionine; and
b) operating the simple culture vessel to provide an oxygen transfer rate greater than 2.0 millimoles O₂/liter/min.

45. The method of claim 44, wherein the medium comprises: 0.04-4% glucose, NH₄Cl at a concentration of 20-60 mM, KH₂PO₄ at a concentration of 20-60 mM, Na₂HPO₄ at a concentration of 75-115 mM, Na₂HPO₄-7H₂O at a concentration of 75-115 mM, MgSO₄ at a concentration of 0.5-4 mM, FeSO₄ at a concentration of 70-110 mM, CaCl₂ at a concentration of 80-120 μM, one or more amino acids but not methionine, vitamins, and seleno-L-methionine at a concentration of 100-300 μM.

46. The method of claim 44, wherein the simple culture vessel is a Tunac-type flask.

47. A method for high-throughput production of polypeptides, the method comprising:
a) obtaining a plurality of cell lines, each cell line comprising an exogenously controlled expression construct for expressing a nucleic acid encoding a polypeptide;
b) generating a plurality of cell cultures by placing in separate simple culture vessels
i) medium suitable for growth of a high density cell culture, and
ii) cells of one of the plurality of cell lines; and
c) operating the simple culture vessels to provide an oxygen transfer rate suitable for growth of a high density cell culture, thereby obtaining a high density cell culture having an OD₆₀₀ of 4 or greater; and
d) obtaining a purified polypeptide composition from a plurality of the high density cell cultures, thereby obtaining purified polypeptide compositions.

48. The method of claim 47, wherein each cell line comprises an exogenously controlled expression construct for expressing a nucleic acid encoding a polypeptide, wherein each polypeptide is a polypeptide of the proteome of a subject organism.

49. The method of claim 47, wherein each cell culture is contacted with an inducer at an OD₆₀₀ of 1 or greater.

50. The method of claim 47, wherein each cell culture is contacted with inducer at approximately the same time after generating the cell culture.

51. A cell culture medium comprising:
a) tryptone at a concentration of 10-14 grams/L;
b) yeast extract at a concentration of 20-30 grams/L;
c) buffering salts at an initial pH between 6 and 8;
d) a metal mixture comprising: cobalt, manganese, copper, boron, molybdenum, zinc, iron, calcium, aluminum and nickel; and
e) a magnesium source.

52. The cell culture medium of claim 51, wherein the magnesium source is MgSO₄.

53. The cell culture medium of claim 51, the medium further comprising 0.5-5% glycerol.

54. The cell culture medium of claim 51, wherein the metal mixture comprises: CoCl₂-6H₂O (4-8 μM), MnSO₄-5H₂O (20-40 μM), CuCl₂-2H₂O (4-8 μM), H₃BO₃ (6-10 μM), Na₂MoO₄-2H₂O (6-10 μM), ZnSO₄-7H₂O (4-8 μM), FeSO₄-7H₂O (75-125 μM), CaCl₂-2H₂O (40-100 μM), AlCl₃-6H₂O (2-6 μM), and NiCl₂-6H₂O (6-10 μM).

55. The cell culture medium of claim 51, wherein the buffering salts comprise potassium and phosphate, the phosphate at a concentration of between 100 and 200 mM.

56. A dry composition that, when mixed with an appropriate volume of water, provides a cell culture medium of any of claim 51-55.

57. A concentrated medium that, when mixed with an appropriate volume of water, provides a cell culture medium of any of claim 51-55.

58. A metal mixture comprising water and at least eight elements selected from the group consisting of: cobalt, manganese, copper, boron, molybdenum, zinc, iron, calcium, aluminum and nickel.

59. A metal mixture of claim 58 having a pH between 6 and 8.

60. A metal mixture of claim 58, comprising cobalt, manganese, copper, boron, molybdenum, zinc, iron, calcium, aluminum and nickel.

61. The metal mixture of claim 58, comprising: CoCl₂-6H₂O (4-8 μM), MnSO₄-5H₂O (20-40 μM), CuCl₂-2H₂O (4-8 μM), H₃BO₃ (6-10 μM), Na₂MoO₄-2H₂O (6-10 μM), ZnSO₄-7H₂O (4-8 μM), FeSO₄-7H₂O (75-125 μM), CaCl₂-2H₂O (40-100 μM), AlCl₃-6H₂O (2-6 μM), and NiCl₂-6H₂O (6-10 μM).

62. A dry composition that, when mixed with an appropriate volume of water, provides a metal mixture of any of claims 58-61.

63. A concentrated metal mixture that, when mixed with an appropriate volume of water, provides a metal mixture of any of claims 58-61.
